(19) United States
(12) Patent Application Publication
(10) Pub. No.: US 2007/0258991 A1
(43) Pub. Date: Nov. 8, 2007

(54) IMMUNOGENIC AGENT THERAPY USING PLASMAPHERESIS

(75) Inventors: Pete T. Buasen, Jefferson, MD (US); Sylvain Cardin, Middletown, MD (US); Robert M. Lorence, Bethesda, MD (US)

Correspondence Address: LEWIS J. KREISLER, LEGAL DEPARTMENT, 930 CLOPPER ROAD, GAITHERSBURG, MD, 20878, US

(73) Assignee: WELLSTAT BIOLOGICS CORPORATION, Gaithersburg, MD (US)

(21) Appl. No.: 11/677,853

(22) Filed: Feb. 22, 2007

Related U.S. Application Data
(62) Division of application No. 10/512,760, filed on Oct. 26, 2004.
(60) Provisional application No. 60/375,931, filed on Apr. 26, 2002.

(51) Int. Cl. A61K 39/00

(52) U.S. Cl. 424/184.1

(57) ABSTRACT

Lowering the level of antibody or complement in the blood of a subject by plasmapheresis or exchange transfusion prior to administering an immunogenic therapeutic agent containing a foreign epitope reduces the immune response of the subject to the therapeutic agent.

### FIELD OF THE INVENTION

This invention is in the field of therapies utilizing therapeutic agents that are immunogenic.

### BACKGROUND OF THE INVENTION

Therapeutic viruses (either oncolytic viruses or replication-incompetent viruses for gene therapy) are used for the treatment of cancer and other diseases (Kim, et al., Trends Mol. Med., 8(4) (Suppl.):S68-S73, 2002 (review)). Therapeutic bacteria such as Salmonella are being used as anticancer agents as well (Low, et al., Nat. Biotech., 17:37-41, 1999; Bermudes, et al., Curr. Opin. Drug Discov. Devel., 5(2):194-199, Mar. 5, 2002).

One issue that has arisen is the effects of the immune response on the efficacy of these approaches (Zwiebel, Seminars in Oncology, 28(4):336-343 at 338, left column, 2001). Depending upon the agent, antibodies might be pre-existing, such as the case for most adenovirus strains. In one study, neutralizing antibodies to adenovirus type 5 were found in 60% of normal controls and in 46% of prostate cancer patients (Chen, et al., Hum. Gene Ther., 11:1553-1567, 2000). For other agents such as Newcastle disease virus ( Pecora et al., J. Clin. Oncol., In Press, May 1, 2002 scheduled) or Vesicular stomatitis virus, pre-existing antibodies in the general North America population are rare. However, use of these agents induces an immune response in mammals (Pecora, ibid.).

Cobra venom factor has been used to facilitate infection by blocking the effects of complement and thereby reducing the immune response (Ikeda, et al., J. Virol., 74(10): 4765-4775, 2000). However the use of cobra venom is not a practical solution.

Chen, ibid., in discussing the issue of pre-existing antibodies to adenovirus, proposed an immunoapheresis technique using an affinity column to specifically lower the level of adenovirus antibodies from the patient prior to treatment. However, this method is expensive and cumbersome, in part because a column containing virus-specific antigens must be generated. It also does not remove complement, which may also have a negative impact on the therapy. Immunoapheresis, also known as immunoadsorption, is distinct from plasmapheresis (Schneider, “Plasmapheresis and immunoadsorption: Different techniques and their current role in medical therapy”, Kidney Int'l, 53(Suppl. 64):S61-S65, 1998)

Other means to reduce the antibody response in patients to viral therapy include the use of immunsuppresive agents (Todo, et al., Hum. Gene Ther., 10:2869-2878, 1999). The two main drawbacks of this approach are (1) the reduction in a beneficial cellular immunity in the case of cancer treatment (Todo, ibid.) and (2) the increased risk of infection of normal tissue by either the replication competent agent itself or by an opportunistic infection.

Plasmapheresis has been used to treat a number of autoimmune diseases (Schneider, ibid.). Nevertheless, although the problem of reduced efficacy of virus-based therapies resulting from the immune response has been recognized for a number of years (Schulick, et al., J. Clin. Invest., 99(2):209-219, 1997), plasmapheresis has not previously been applied to enhance the efficacy of such therapies.

### SUMMARY OF THE INVENTION

This invention provides a method of reducing the immune response to an immunogenic therapeutic agent in a subject to whom the agent is administered wherein the agent contains at least one epitope foreign to the subject, comprising treating the subject with a blood antibody-depletion technique selected from the group consisting of plasmapheresis and exchange transfusion to lower the level of antibody or complement in blood of the subject prior to administering the agent.

The method of this invention improves existing therapeutic approaches that utilize an immunogenic therapeutic agent by providing a technique for reducing the immune response during treatment, while avoiding some of the drawbacks associated with immunosuppression and immunoapheresis.

### BRIEF DESCRIPTION OF THE FIGURE

FIG. 1: Mean anti-PPMK107 neutralizing antibody titers in mice after blood exchange

### DETAILED DESCRIPTION OF THE INVENTION

In accordance with this invention any conventional therapeutic agent that is immunogenic and contains a foreign epitope can be utilized. In one embodiment of this invention the therapeutic agent is a therapeutic virus, for example an oncolytic virus, an adenovirus, or a herpes virus. Examples of oncolytic viruses that can be utilized in accordance with this invention include a Newcastle Disease Virus, a Vesicular Stomatitis Virus, and a reovirus. The use of oncolytic viruses is disclosed in WO 00/62735 and WO 01/19380, the contents of which are incorporated herein by reference. Adenovirus has been used in gene therapy and as an oncolytic virus. In another embodiment the therapeutic agent is bacterial. Examples of bacterial therapeutic agents that can be utilized in accordance with this invention include a Salmonella bacteria, a Clostridium bacteria, or a Bifido bacteria. Salmonella typhimurium is a preferred Salmonella bacteria.

After reduction of antibody and complement levels in the blood, the levels of antibody and complement gradually recover. The amount of time for such recovery depends on a number of factors, including the individual patient and the amount of antibody and complement removed by the plasmapheresis procedure. In accordance with this invention the amount of time between plasmapheresis and administration of the therapeutic agent is selected such that the levels of antibody or complement in the blood at the time of administration are lower than the levels prior to plasmapheresis. Generally the plasmapheresis is performed up to twenty-four hours, preferably up to six hours, most preferably up to one hour before administration of the therapeutic agent.

In accordance with this invention, any conventional method of plasmapheresis can be utilized. Plasmapheresis is a process in which plasma, the fluid part of the blood, is removed from the blood cells by a cell separator. The cell separator works by either centrifugation or by filtration. The cells are then returned to the person undergoing plasmapheresis, while the plasma is typically discarded and replaced by other fluids such as new plasma from different source(s) or a colloid solution such as 5% albumin or synthetic plasma expanders (Reimann and Mason, 1990). The term “plasma exchange” is more commonly applied to the removal of larger volumes of plasma (>1 L), although the term plasmapheresis is also used in this situation (Reimann and Mason, Intensive Care Med., 16:3-10, 1990 (review); and Patten, in CRC Critical Reviews in Clin. Lab. Sciences, 23(2):147-175). As used herein the term “plasmapheresis” includes both plasmapheresis and plasma exchange.

Plasmapheresis, in addition to removing antibodies, can also be used to lower the level of complement. Complement may also have a negative impact on therapy with therapeutic viruses or bacteria. In the case of plasmapheresis by filtration, the patient's own plasma can be returned after depletion of plasma proteins in the size range of immunoglobulins. Plasmapheresis using plasma filters has an added advantage over plasmapheresis using centrifugation in being more cost effective (since the patient's own plasma can be returned) and in not causing deficiency syndromes (e.g., depletion of clotting factors; Siami, et al., ASAIO J., 46:383-8, 2000).

In one embodiment of this invention the plasmapheresis comprises: (a) obtaining from the subject blood which comprises cells and plasma, which plasma comprises antibodies or complement; (b) centrifuging the blood to isolate the plasma from the cells; and (c) returning the cells to the subject.

In another embodiment the plasmapheresis comprises: (a) obtaining from the subject blood which comprises cells and plasma, which plasma comprises antibodies or complement; (b) filtering the blood with a first filter to separate the plasma from the cells; and (c) returning the cells to the subject. Preferably the filter used to separate plasma from cells has a size cut-off of from 0.1 to 0.6 microns. A more specific embodiment that returns the plasma to the subject comprises filtering the plasma isolated in step (b) is with a second filter to deplete antibody or complement from the plasma and returning the depleted plasma to the subject. Preferably the second filter has a molecular weight cut-off of from 60 to 150 kilodaltons.

In accordance with this invention any convention method for performing exchange transfusion to lower the level of antibody or complement in the blood of the subject prior to administering the immunogenic agent. (For example, see Looareesuwan, et al., Q. J. Med., New Series 75, No. 277, pp. 471-481, May 1990; and Adamkin, Ped. Clin. N. Amer., 24(3): 599-604, August 1977.) In exchange transfusion, the patient's blood is removed and, at the same time, replaced with donor blood (Sacher R A and Lenes B A, 1981). Exchange transfusion is a method that, like plasmapheresis, exchanges or replaces blood plasma. Unlike plasmapheresis, the other blood components are also exchanged in this method. Exchange transfusion have been used to treat neonates with high levels of bilirubin in the blood (Peterec, in Perinatal Hematology, 22(3): 561-592, September 1995;) and to treat malaria (Phillips et al., Rev. Infect. Dis., 12(6): 1100-1108, 1990; Elder et al., Scot. Med. J., 35: 148-149, 1990) with the aim of removing bilirubin and the malaria parasite, respectively. Examples of exchange transfusion to lower the antibodies toward an immunogenic agent are given in Examples 3 and 4. As used herein the expressions “exchange transfusion” and “blood exchange” are synonymous.

In accordance with this invention the subject can be a human or a non-human mammal.

The invention described herein will be better understood by reference to the following examples. The examples are illustrative only, and do not limit the invention defined by the claims. In the following examples the NDV used was a triple-plaque purified (PP) attenuated (mesogenic) version of the MK107 strain of Newcastle disease virus, described more fully in International Patent Publication WO 00/62735, published Oct. 26, 2000 (Pro-Virus, Inc.). The entire content of WO 00/62735 is hereby incorporated herein by reference.

### EXAMPLES

### Example 1

A cancer patient receives three courses of an attenuated Newcastle disease virus. Each of these three first courses consist of six total treatments given at three times per week for two weeks followed by a one week rest period. For each course, a first dose of 1 billion PFU/m² is given followed by a second dose of 12 billion PFU/m² and four doses of 24 to 120 billion PFU/m². Before the patient's fourth course, at a time for which the patient has developed antibodies to virus, the patient undergoes plasmapheresis using filtration or centrifugation. Within one hour of completing the plasmapheresis, the patient is treated with a dose of 1 to 12 billion PFU/m². Within the following week, the patient receives two more doses ranging from 12 to 120 billion PFU/m².

### Example 2

A cancer patient receives three courses of an attenuated Newcastle disease virus. Each of these three first courses consist of six total treatments given at three times per week for two weeks followed by a one week rest period. For each course, a first dose of 24 billion PFU/m² administered over 3 hours is given followed by a five doses of 120 billion PFU/m². Before the patient's fourth course, at a time for which the patient will have developed antibodies to virus, the patient undergoes plasmapheresis using filtration or centrifugation. Within one hour of completing the plasmapheresis, the patient is treated with a dose ranging from 24 to 120 billion PFU/m² administered over 3 hours. Within the following week, the patient receives two more doses of 120 billion PFU/m²

### Example 3

Exchange Transfusion of Pre-Immunized Mice Reduces the Levels of Neutralizing Antibodies to Newcastle Disease Virus PPMK107 in Serum.

Immunization.

Female C3H/Hen mice were given intravenous doses of 1E+09 PFU of PPMK107 (an attenuated Newcastle disease virus described in WO 00/62735) weekly for at least 4 weeks to generate neutralizing antibodies in the serum against PPMK107 and were therefore pre-immunized to PPMK107

Catheter Implantation:

Mice were anesthetized with ketamine/xylazine and their surgical site shaved (neck and thoracic region; back of neck). Implantation of the catheter was performed by accessing the carotid artery with polyethylene tubing inserted into the lumen of the artery and attached with three silk ligatures to keep the tube in place. After successful implantation of the catheter, it was exteriorized between the scapulae. Before being capped, the catheter was filled with a solution of heparin. The mouse was given 3 days or more to recover from the surgery before the blood exchange was performed.

Blood Exchange:

In preparation for blood exchange, heparinized naive donor blood was collected from the same strain of mice (C3H/Hen mice).

The exchange started with the mouse exposed to a heat lamp (particularly the tail) to dilate the tail vein in preparation for IV catheter tube insertion as the method for infusing the donor blood into the mouse. As soon as the tail was ready for tail IV, the catheter was inserted into the tail vein and then the mouse anesthetized with ketamine/xylazine. When the mouse was immobilized, the indwelling catheter was uncapped and the content aspirated. The catheter was connected to atubing that reach the blood collection tube. As soon as blood started to flow (one to two drops was allowed to drip to remove any blood clot or heparin remnant), the desired sample (pre-bleed sample) was collected first before the actual blood exchange process had begun. The exchange started with injection of donor blood slowly about 1.0 ml of blood in every 2.5 minutes. Blood was collected in a continuous manner after the completion of each ml blood infused (depending on the desired blood sample). A total of 5 to 6 ml of donor blood was infused and 4.5 to 5.5 ml of blood was taken out from the mouse. After completion of blood exchange, the catheter was flushed with heparin before it was plugged. Serum was collected from each blood sample after low speed centrifugation.

Microplate Assay for PV701 Neutralizing Antibody In Mouse Serum Samples

Anti-PPMK107 serum (assay control) was serially diluted in assay buffer (DMEM with 4.5 g/L Glucose, 25 mM HEPES, 2% FBS, 2 mM L-Glutamine, 100 U/L Penicillin, and 100 μg/ml Streptomycin) across the 12 columns of duplicate rows of a 96 plate (starting at 1:36.75 in the first column and performing 1:3.5 serial dilutions). Samples (unknowns) were heat inactivated for 30 minutes at 56° C., diluted 1:10.5 into the first column of the plate (in triplicate) and then diluted 1:3.5 across the plate in assay buffer. The total sample volume after dilutions were performed was 75 μl/well. PPMK107 was diluted to a concentration of 2.8E+5 PFU/ml. 40 μl were added to each well (11,200 PFU/well). The plates were incubated for 2 hours (at 37° C.) to allow the antibody (if present) to interact with the virus. HT1080 human fibrosarcoma cells were then added (40 μl containing 5000 cells) to each well and the plates were incubated for 68-72 hours. Quantitative assessment of cell viability was performed using MTS. 40 μl of MTS were added to each well, and plates were incubated (at 37° C.) for 2 hours. The amount of signal is directly proportional to the number of viable cells in the well. The reaction was stopped by adding 20 μl of a 10% SDS solution to each well. Absorbance values were read in a microplate spectrophotometer at 490 nm. Each dilution series was plotted using a 4 parameter logistics (4-PL) fit and the midpoint or TC50 was calculated. The anti-PPMK107 serum (assay control) was used to show reproducibility between plates in the assay. The TC50 for each sample was reported to demonstrate the relative differences in the PPMK107 neutralizing antibody levels in the samples.

Results

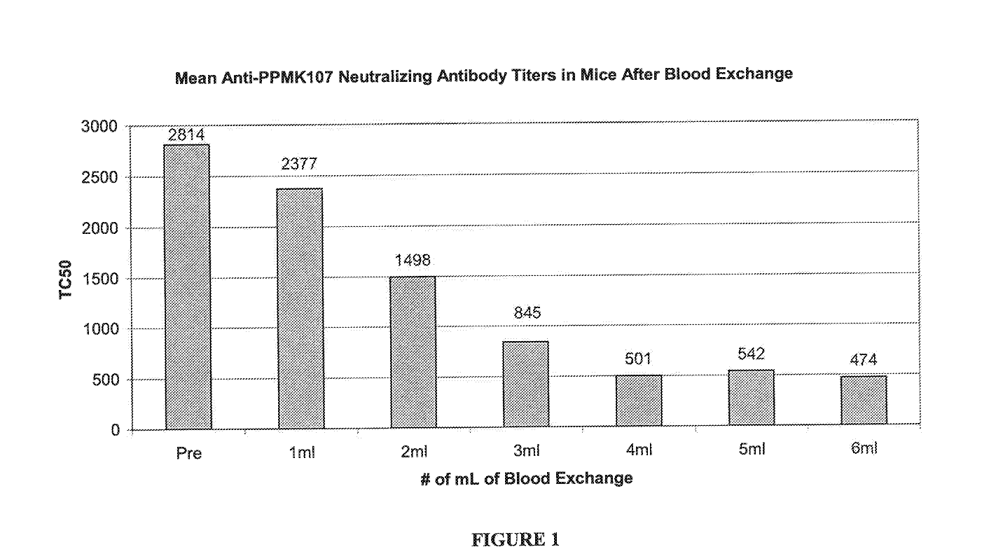

Nine mice had blood exchanges performed. In FIG. 1, the mean antibody titers against PPMK107 are shown. With each ml of exchange, the antibody titer decreased from baseline and reached a lower level after approximately 4 ml of exchange. This demonstrates that exchange of plasma-containing blood with the equivalent blood component from donors with undetectable antibody titers can decrease the level of antibodies measured in the serum.

For repeat courses after neutralizing antibodies to PPMK107 develop, intravenous therapy against tumors in immune competent mice such as C3H/Hen using PPMK107 is made more effective following this exchange transfusion. Mice are dosed with PPMK107 (dose range per mouse of 1E+08 to 1E+09 PFU) immediately after the blood exchange occurs. Additional blood exchanges and followed immediately by repeat dosing are repeated to achieve the maximal antitumor effect.

### Example 4

Effect of Exchange Transfusion in Conscious Pre-Immunized Mice on Neutralization of Antibodies to Newcastle Disease Virus PPMK107 in Serum.

In this example catheters were inserted in the carotid artery and jugular vein. The advantage of this technique is that the mice were conscious, which is often convenient.

Immunization.

Female C3H/Hen mice were given intravenous doses of 1E+09 PFU of PPMK107 (an attenuated Newcastle disease virus described in WO 00/62735) weekly for at least 4 weeks to generate neutralizing antibodies in the serum against PPMK107 and were therefore pre-immunized to PPMK107

Catheter Implantation:

Mice were anesthetized with ketamine/xylazine and their surgical site shaved (neck and thoracic region; back of neck). Implantation of the catheters was performed by accessing the carotid artery and the jugular vein with polyethylene and silastic tubing respectively inserted into their lumen and attached with three silk ligatures to keep the catheters in place. After successful implantation of the catheters, they were exteriorized between the scapulae. Before being capped, the catheters were filled with a solution of heparin. The mice were given 3 days or more to recover from the surgery before the blood exchange was performed.

Blood Exchange:

In preparation for blood exchange, heparinized naive donor blood was collected from the same strain of mice (C3H/Hen mice).

The exchange started after the indwelling catheters were uncapped and the content aspirated. The arterial catheter was connected to a tubing that reach the blood collection tube. The veinous catheter was connected to a tubing that is in turn connected to a syringe containing the donor blood for the blood exchange. As soon as blood started to flow from the arterial catheter (one to two drops were allowed to drip to removed any blood clot or heparin remnant), the desired sample (pre-bleed sample) was collected first before the actual blood exchange process began. The exchange started with injection of donor blood slowly about 1.0 ml of blood in every 2.5 minutes. Blood was collected in a continuous manner after the completion of each millileter of blood infused (depending on the desired blood sample). A total of 5 to 6 ml of donor blood was infused and 4.5 to 5.5 ml of blood was taken out from the mice. After completion of blood exchange, the catheters were filled with heparin before they were recapped. Serum was collected from each blood sample after low speed centrifugation.

(Of eight mice undergoing the preceding procedure four died from the surgery. Because of time constraints catheterization of a tail vein was used instead of jugular/carotid catheterization. Nevertheless, because similar surgeries have gone well in other ongoing projects, jugular/carotid catheterization is still useful because it allows treatment of a conscious subject.)

Blood from animals undergoing the surgery described in this example was not tested for antibody titer. However the blood can be tested in accordance with the Microplate Assay for PV701 Neutralizing Antibody In Mouse Serum Samples described in Example 3.

What is claimed is:

1. A method of reducing the immune response to an immunogenic therapeutic agent in a subject to whom the agent is administered wherein the agent contains at least one epitope foreign to the subject, comprising treating the subject with plasmapheresis to lower the level of antibody or complement in blood of the subject prior to administering the agent;
wherein the plasmapheresis comprises:
(i) (a) obtaining from the subject blood which comprises cells and plasma, which plasma comprises antibodies or complement; (b) centrifuging the blood or filtering the blood with a filter, to isolate the plasma from the cells; and (c) returning the cells and not the plasma to the subject;
or
(ii) (a) obtaining from the subject blood which comprises cells and plasma, which plasma comprises antibodies or complement; (b) filtering the blood with a first filter to separate the plasma from the cells;
(c) returning the cells to the subject; (d) size-filtering the plasma isolated in step (b) with a second filter to deplete antibody or complement from the plasma; and (e) returning the depleted plasma to the subject.

2. The method of claim 1, wherein the agent is a therapeutic virus.

3. The method of claim 2, wherein the virus is an oncolytic virus.

4. The method of claim 3, wherein the oncolytic virus is a Newcastle Disease Virus.

5. The method of claim 3, wherein the oncolytic virus is a Vesicular Stomatitis Virus.

6. The method of claim 3, wherein the oncolytic virus is a reovirus.

7. The method of claim 2, wherein the virus is an adenovirus or a herpes virus.

8. The method of claim 1, wherein the agent is bacterial.

9. The method of claim 8, wherein the bacterial therapeutic agent is a Salmonella.

10. The method of claim 9, wherein the agent is a Salmonella typhimurium.

11. The method of claim 8, wherein the bacterial therapeutic agent is a Clostridium.

12. The method of claim 8, wherein the bacterial therapeutic agent is a Bifidobacterium.

13. The method of claim 1, wherein the plasmapheresis is performed up to twenty-four hours before administration of the agent.

14. The method of claim 13, wherein the plasmapheresis is performed up to six hours before administration of the agent.

15. The method of claim 14, wherein the plasmapheresis is performed up to one hour before administration of the agent.

16. The method of claim 1, wherein the plasmapheresis comprises: (a) obtaining from the subject blood which comprises cells and plasma, which plasma comprises antibodies or complement; (b) centrifuging the blood to isolate the plasma from the cells; and (c) returning the cells and not the plasma to the subject.

17. The method of claim 1, wherein the plasmapheresis comprises: (a) obtaining from the subject blood which comprises cells and plasma, which plasma comprises antibodies or complement; (b) filtering the blood with a filter to separate the plasma from the cells; and (c) returning the cells and not the plasma to the subject.

18. The method of claim 17, wherein the filter has a size cut-off of from 0.1 to 0.6 microns.

19. The method of claim 16 or 17, further comprising infusing into the bloodstream of the subject a plasma-replacement fluid in an amount approximately equivalent in volume to the plasma resulting from step (b), wherein the plasma-replacement fluid is not the plasma resulting from step (b) and the infusion is performed after any one or more of steps (a), (b) or (c).

20. The method of claim 19, wherein the plasma-replacement fluid is plasma from a source other than the subject.

21. The method of claim 1, wherein the plasmapheresis comprises: (a) obtaining from the subject blood which comprises cells and plasma, which plasma comprises antibodies or complement; (b) filtering the blood with a first filter to separate the plasma from the cells; (c) returning the cells to the subject; (d) size-filtering the plasma isolated in step (b) with a second filter to deplete antibody or complement from the plasma; and (e) returning the depleted plasma to the subject.

22. The method of claim 21, wherein the first filter has a size cut-off of from 0.1 to 0.6 microns.

23. The method of claim 21, wherein the second filter has a molecular weight cut-off of from 60 to 150 kilodaltons.

24. The method of claim 1, wherein the subject is a human.

25. The method of claim 1, wherein the subject is a non-human mammal.
